(19) United States
(12) Patent Application Publication
(10) Pub. No.: US 2007/0173907 A1
(43) Pub. Date: Jul. 26, 2007

(54) Hybrid electrode for a plasma arc torch and methods of manufacture thereof

(75) Inventors: Nakhleh A. Hussary, West Lebanon, NH (US); Thierry R. Renault, West Lebanon, NH (US); William M. Wakefield SR., Windsor, VT (US); Gene V. Hewes, Plainfield, NH (US); Christopher J. Conway, Wilmot, NH (US); Darrin H. MacKenzie, Windsor, VT (US)

Correspondence Address: HARNESS, DICKEY, & PIERCE, P.L.C, 7700 BONHOMME, STE 400, ST. LOUIS, MO, 63105, US

(73) Assignee: Thermal Dynamics Corporation

(21) Appl. No.: 11/340,066

(22) Filed: Jan. 26, 2006

(51) Int. Cl. A61N 1/00

(52) U.S. Cl. 607/61

(57) ABSTRACT

Methods of forming an electrode for use in a plasma arc torch are provided that generally include forming an electrode body defining a proximal end portion and a distal end portion, forming a recess within the distal end portion, securing a slug within the recess of the distal end portion, creating a bore through the slug and the distal end portion of the electrode body, and securing an emissive insert within the bore. In another form, a hybrid electrode is provided, which is formed by the manufacturing methods of the present invention, and which includes an electrode body defining a cavity, a secondary body secured to the electrode body, and an emissive element secured within the bore and in contact with the secondary body and the electrode body, wherein the secondary body is not exposed to the cavity.

### FIELD

The present disclosure relates to plasma arc torches and more particularly to electrodes for use in plasma arc torches.

### BACKGROUND

The statements in this section merely provide background information related to the present disclosure and may not constitute prior art.

Plasma arc torches, also known as electric arc torches, are commonly used for cutting, marking, gouging, and welding metal workpieces by directing a high energy plasma stream consisting of ionized gas particles toward the workpiece. In a typical plasma arc torch, the gas to be ionized is supplied to a distal end of the torch and flows past an electrode before exiting through an orifice in the tip, or nozzle, of the plasma arc torch. The electrode has a relatively negative potential and operates as a cathode. Conversely, the torch tip constitutes a relatively positive potential and operates as an anode during piloting. Further, the electrode is in a spaced relationship with the tip, thereby creating a gap, at the distal end of the torch.

In operation, a pilot arc is created in the gap between the electrode and the tip, often referred to as the plasma arc chamber, which heats and subsequently ionizes the gas. The ionized gas is blown out of the torch and appears as a plasma stream that extends distally off the tip. As the distal end of the torch is moved to a position close to the workpiece, the arc jumps or transfers from the torch tip to the workpiece with the aid of a switching circuit activated by the power supply. Accordingly, the workpiece serves as the anode, and the plasma arc torch is operated in a “transferred arc” mode.

Plasma arc torches typically include several “consumable” components, which generally refer to components that require regular replacement due to wear under relatively extreme operating conditions. Among these consumable components is the electrode, which acts as the cathodic side of the power supply and the source of initiation for the plasma arc and often undergoes the most extreme operating conditions. As the electrode wears after extended periods of operation, the stability of the plasma arc begins to degrade and affects the wear of the other components such as the tip and the shield cap, and thus the quality of the cut correspondingly becomes worse. The more frequently consumable components have to be replaced, the more end users or operators are faced with decreased productivity and increased costs.

Accordingly, many approaches have been developed to improve the life of electrodes such as altering gas flow patterns, and incorporating geometry to enhance cooling, among many others. These approaches have resulted in better quality cuts and lower operating costs for operators, yet still, additional advancements in the art are always desired to further increase the life of consumable components, and especially electrodes, to provide even more cost savings and improved quality cuts for the end user. Many of these approaches used to date, however, have involved relatively expensive materials and manufacturing processes and thus more cost effective methods of manufacturing electrodes exhibiting improved life are still needed in the art of plasma arc torches.

### SUMMARY

In one form of the present invention, a method of fabricating an electrode for use in a plasma arc torch is provided that comprises forming an electrode body having a proximal end portion and a distal end portion, forming a recess within the distal end portion of the electrode body, brazing a slug within the recess of the electrode body, forming at least one face around the distal end portion of the electrode and a distal end face of the slug, creating a bore through the distal end face of the slug such that the bore extends through the slug and the electrode body, and securing an emissive element within the bore, the emissive element being in contact with the slug and the electrode body.

In another form, a method of fabricating an electrode for use in a plasma arc torch is provided that comprises forming an electrode body having a proximal end portion and a distal end portion, forming a cavity within the proximal end portion of the electrode body, forming a recess within the distal end portion of the electrode body, brazing a slug within the recess of the electrode body, machining the slug and the electrode body to form at least one face around the distal end portion of the electrode body and the slug, creating a bore through a distal face of the slug such that the bore is at least partially exposed to the slug and the electrode body, and fixedly mounting an emissive element within the bore, the emissive element being in contact with the slug and the electrode body and not with the cavity.

Additionally, another method of forming an electrode for use in a plasma arc torch is provided that comprises forming an electrode body defining a proximal end portion and a distal end portion, forming a recess within the distal end portion, securing a slug within the recess of the distal end portion, creating a bore through the slug and the distal end portion of the electrode body, and securing an emissive insert within the bore.

In yet another form, a method of forming an electrode is provided that comprises the step of coining a slug, the slug being adapted for attachment to an electrode body. Preferably, an emissive element is pressed within a bore of the slug to form an electrode subassembly, and the electrode subassembly is secured to a distal end portion of an electrode body to form the electrode.

In still another form, a hybrid electrode for use in a plasma arc torch is provided that comprises an electrode body defining a proximal end portion and a distal end portion. The electrode body comprises a cavity within the proximal end portion, and a secondary body is disposed at the distal end portion of the electrode body. The secondary body defines a distal face and a bore formed through the distal face, and an emissive element is disposed within the bore. The emissive insert is in contact with the secondary body and the electrode body, and the secondary body is not exposed to the cavity.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a proximal perspective view of a hybrid electrode constructed in accordance with the principles of the present invention;

FIG. 2 is a distal perspective view of the hybrid electrode in accordance with the principles of the present invention;

FIG. 3 is a side view of the hybrid electrode in accordance with the principles of the present invention;

FIG. 4 is a cross-sectional view, taken along line 4-4 of FIG. 3, of the hybrid electrode in accordance with the principles of the present invention;

FIG. 5 is an exploded perspective view of an electrode body and a slug constructed in accordance with the principles of the present invention;

FIG. 6 is a distal perspective view of the slug secured within a recess of the electrode body and constructed in accordance with the principles of the present invention;

FIG. 7 is a side view of the electrode body and slug in accordance with the principles of the present invention;

FIG. 8 is a cross-sectional view, taken along line 8-8 of FIG. 7, of the slug secured within the recess and constructed in accordance with the principles of the present invention;

FIG. 9 is an enlarged cross-sectional view of a distal end portion of the hybrid electrode in accordance with the principles of the present invention;

FIG. 10 is an enlarged cross-sectional view of a distal end portion of a second embodiment of a hybrid electrode constructed in accordance with the principles of the present invention;

FIG. 11 is an enlarged cross-sectional view of a distal end portion of a third embodiment of a hybrid electrode constructed in accordance with the principles of the present invention;

FIG. 12 is a flowchart illustrating one method of forming a hybrid electrode in accordance with the principles of the present invention;

FIG. 13 is a flowchart illustrating a second method of forming a hybrid electrode in accordance with the principles of the present invention;

FIG. 14 is a flowchart illustrating a third method of forming a hybrid electrode in accordance with the principles of the present invention;

FIG. 15 is a perspective view of sheet stock defining patterns for a slug to be generated by a coining process in accordance with the principles of the present invention;

FIG. 16 is a perspective view of an electrode subassembly constructed in accordance with the principles of the present invention;

FIG. 17 is a side view of the electrode subassembly in accordance with the principles of the present invention;

FIG. 18 is a cross-sectional view, taken along line 18-18 of FIG. 17, of the electrode subassembly in accordance with the principles of the present invention;

FIG. 19 is a cross-sectional view of the electrode subassembly and the electrode body constructed in accordance with the principles of the present invention; and

FIG. 20 is a flowchart illustrating a fourth method of forming a hybrid electrode in accordance with the principles of the present invention.

### DETAILED DESCRIPTION

The following description is merely exemplary in nature and is not intended to limit the present disclosure, application, or uses. It should be understood that throughout the drawings, corresponding reference numerals indicate like or corresponding parts and features.

In the various forms described herein, relatively low cost methods of manufacturing electrodes that exhibit increased life over conventional electrodes, as well as the other plasma arc torch components, are provided in accordance with the teachings of the present invention. Additionally, different electrode embodiments are also provided, which are produced from the relatively low cost manufacturing methods described herein.

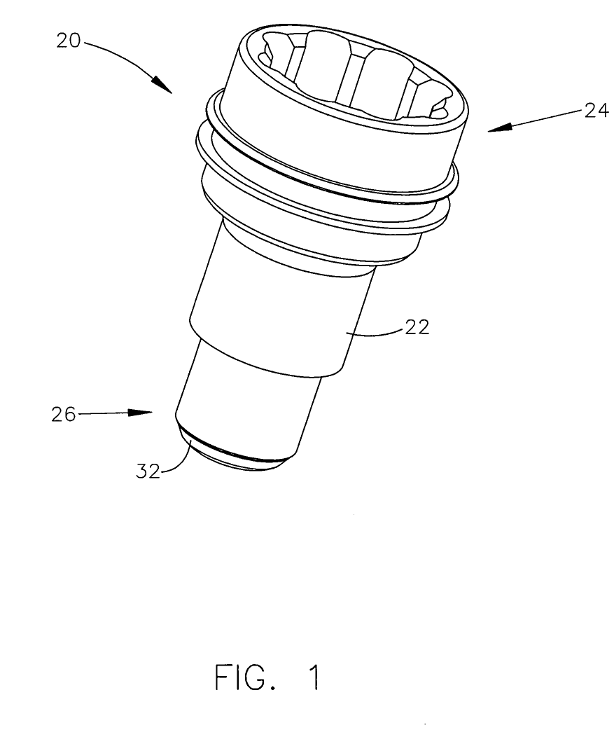

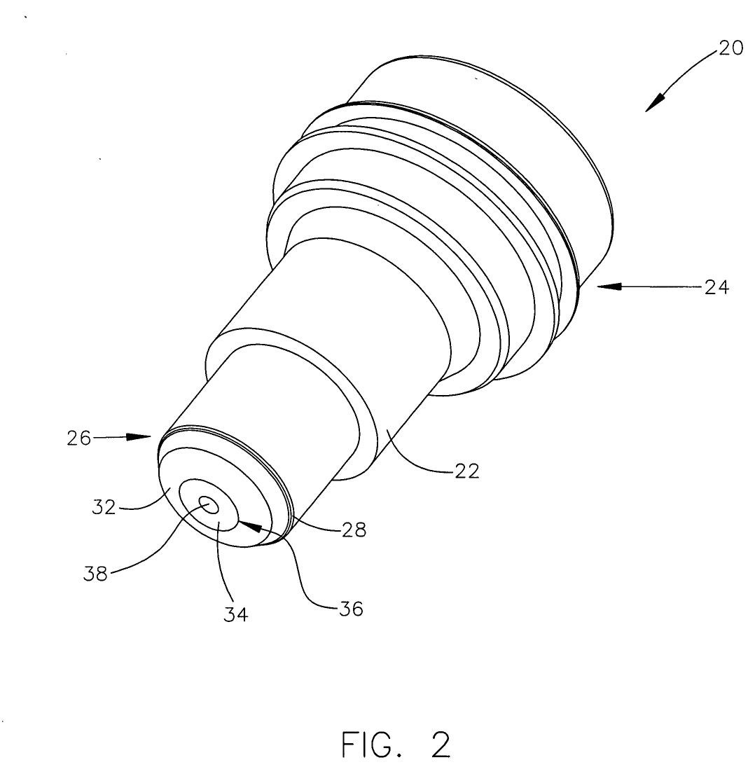

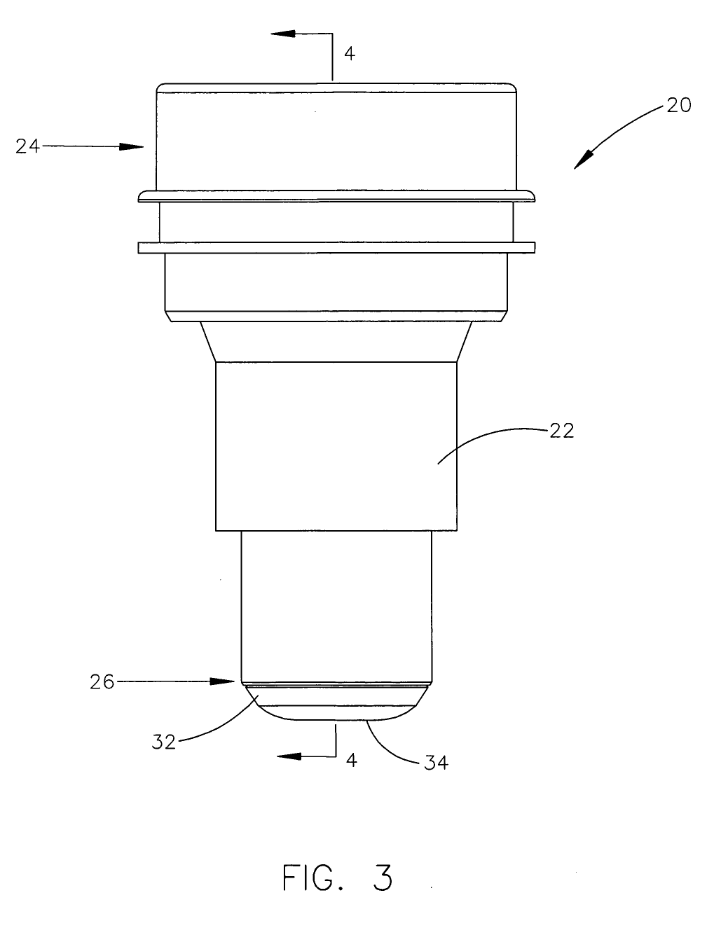

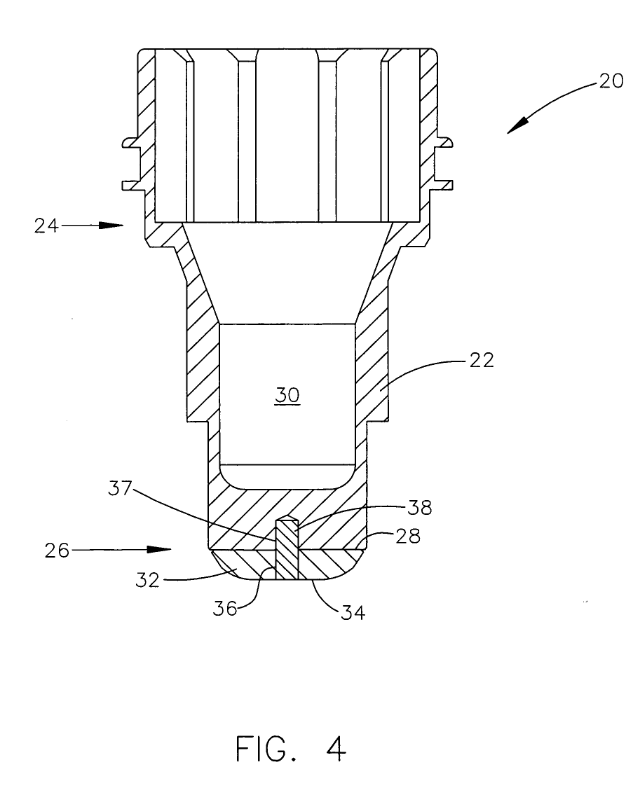

Referring to FIGS. 1-4, a hybrid electrode in one form of the present invention is illustrated and generally indicated by reference numeral 20. The hybrid electrode 20 is operable with a plasma arc torch such as that described in copending application Ser. No 10/409,650, filed Apr. 7, 2003 and titled “Plasma Arc Torch,” which is commonly assigned with the present application and the contents of which are incorporated by reference herein in their entirety.

As shown, the hybrid electrode 20 comprises an electrode body 22 that defines a proximal end portion 24 and a distal end portion 26, wherein the distal end portion 26 further comprises a distal perimeter surface 28. The electrode body 22 also comprises a cavity 30 within the proximal end portion 24, which in general, accommodates a cooling fluid during operation of the plasma arc torch. As further shown, a secondary body 32 is secured to the distal end portion 26 of the electrode body 22 proximate the distal perimeter surface 28. The secondary body 32 defines a distal face 34 and a bore 36 formed through the distal face 34 and preferably all the way through the secondary body 32. An emissive insert 38 is disposed within the bore 36, and the electrode body 22 also includes a bore 37 that is formed conjointly with the bore 36. Accordingly, the emissive insert 38 preferably extends all the way through the secondary body 32 and at least partially through the electrode body 22. In this preferred form, the emissive insert 38 is not exposed to the cavity 30. Alternately, the emissive insert 38 could extend all the way through the electrode body 22 and be exposed to the cavity 30 (not shown) while remaining within the scope of the present invention.

The electrode body 22 is preferably a copper or copper alloy material that provides the requisite electrical conductivity as the electrode 20 comprises a part of the cathodic, or negative, side of the power supply (not shown). The secondary body 32 is preferably a silver or silver alloy material, however, other materials such as gold and gold alloys, among other materials, may also be employed while remaining within the scope of the present invention. The emissive insert 38 is preferably hafnium, however, other materials such as zirconium, tungsten, and tungsten alloys may also be employed while remaining within the scope of the present invention. Generally, the material for the secondary body 32 has a lower thermal emission of electrons than the material for the electrode body 22 such that a plasma arc that is attached to the emissive insert 38 during operation of the plasma arc torch remains attached to the emissive insert 38 and does not have a tendency to attach itself to the electrode body 22, or the secondary body 32, thus reducing overall wear and improving the life of the hybrid electrode 20, and other components of the plasma arc torch.

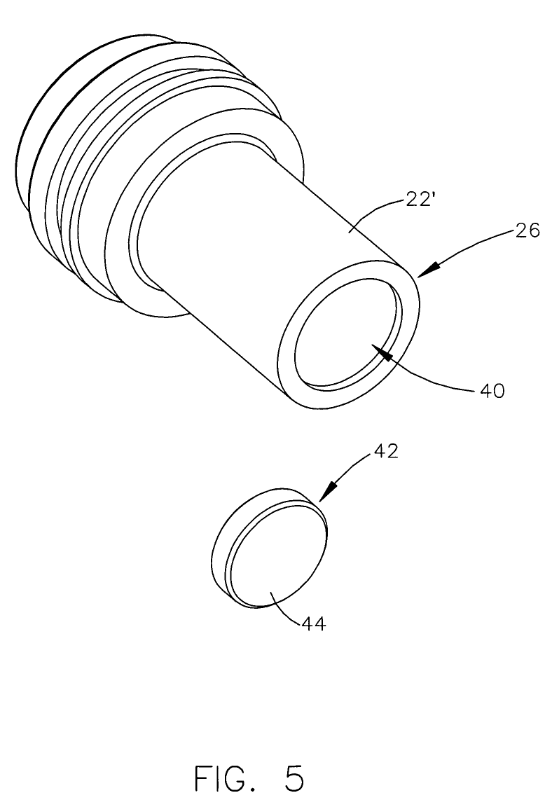

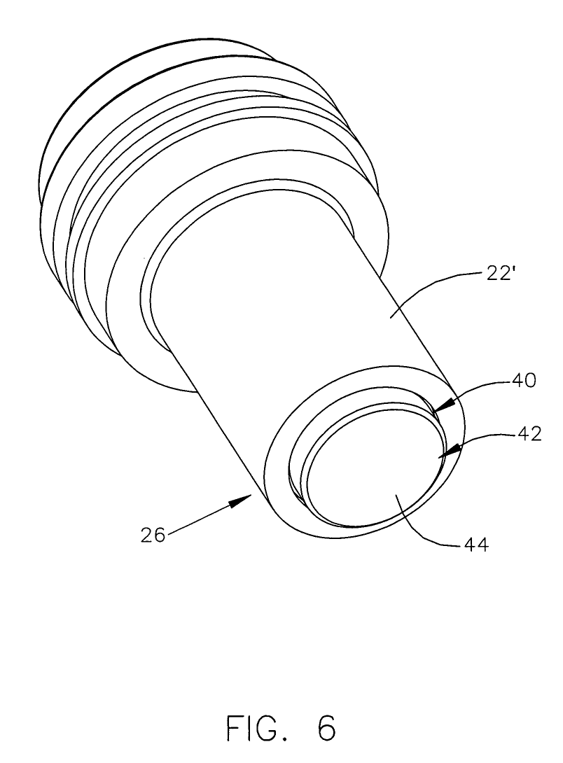

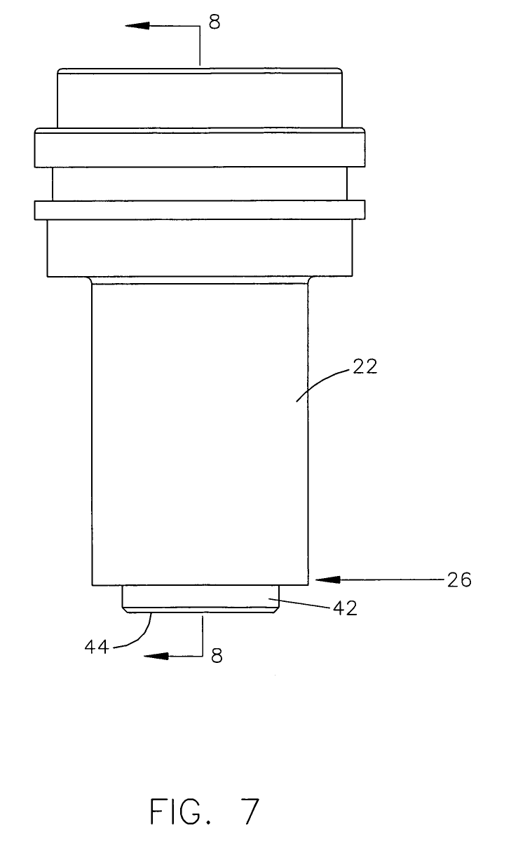

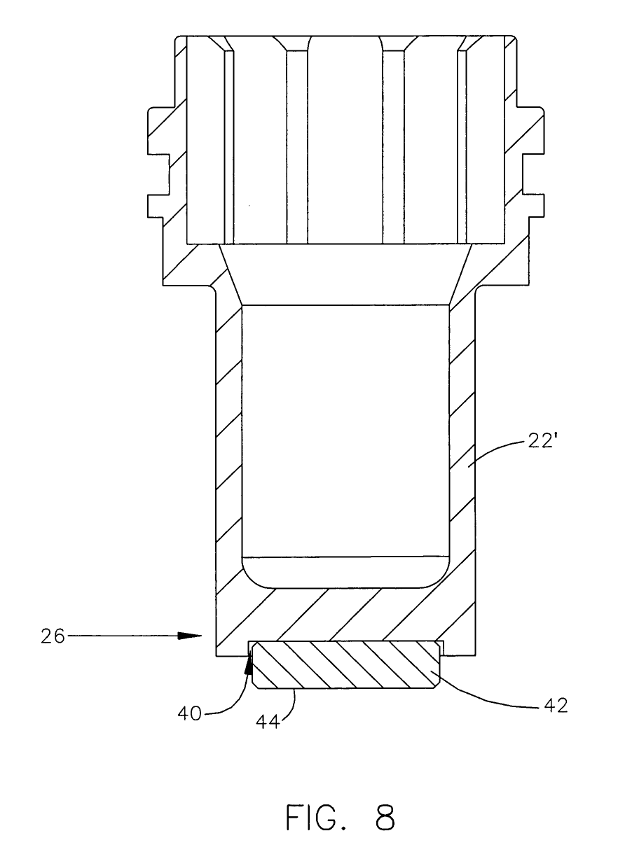

Referring now to FIGS. 5-8, a method of forming the hybrid electrode 20 according to one form of the present invention is now described in detail. As shown, an electrode body pre-form 22′ comprises a recess 40 formed within the distal end portion 26. A slug 42 is provided as a pre-form for the secondary body 32, and the slug 42 is secured within the recess 40 as shown. The slug 42 is sized to fit within the recess 40 and also comprises a distal end face 44 through which the emissive insert 38 is disposed as described in greater detail below.

Preferably, the slug 42 is secured by brazing, however other methods such as mechanical press fitting and swaging, friction welding, mechanical threading, heat treatment, and adhesive bonding, among others, may also be employed while remaining within the scope of the present invention. Additionally, a brazing compound is preferably a silver solder paste, however other compounds such as a silver alloy solder paste, flux and silver solder paste, a flux, among others, may also be employed while remaining within the scope of the present invention.

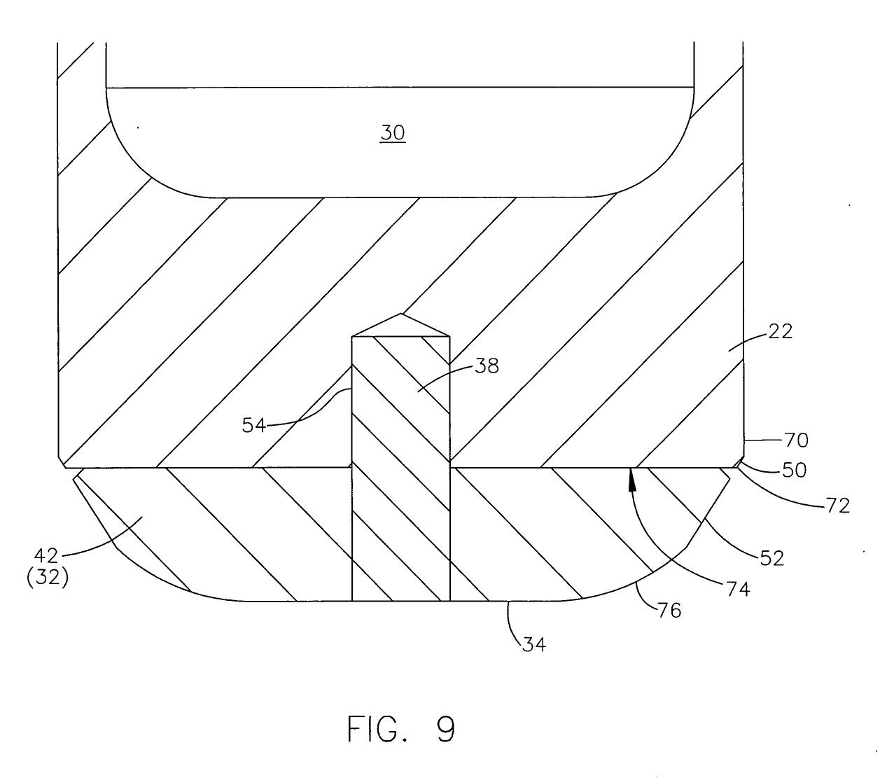

Referring to FIG. 9, after the slug 42 has been secured within the recess 40, a face 50 is formed around the distal end portion 26 of the electrode body 22 and a secondary face 52 is formed around the slug 42. Preferably, the faces 50 and 52 are formed by machining, however, other methods such as metal injection molding, casting, stamping, and molding, among others, may also be employed while remaining within the scope of the present invention. After the faces 50 and 52 are formed, a bore 54 is formed through the distal end face 44 of the slug 42 and the electrode body 22 such that the bore 54 extends all the way through the slug 42 and at least partially through the electrode body 22 as shown. Next, the emissive insert 38 is secured within the bore 54, wherein the emissive insert 38 is in contact with both the slug 42 and the electrode body 22. Preferably, the emissive insert 38 is secured within the bore 54 by press fitting, however, other methods may also be employed while remaining within the scope of the present invention.

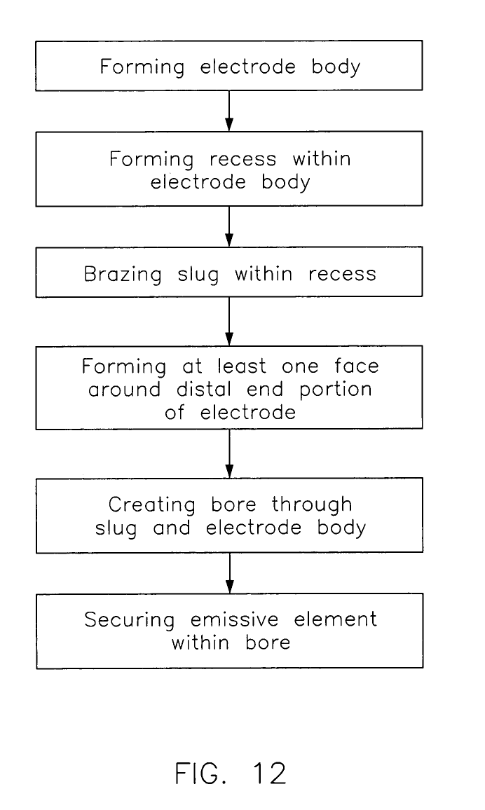

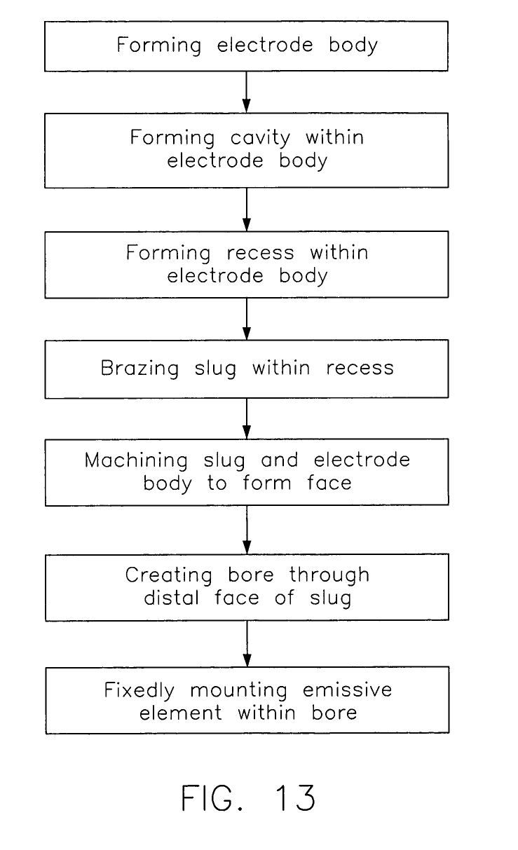

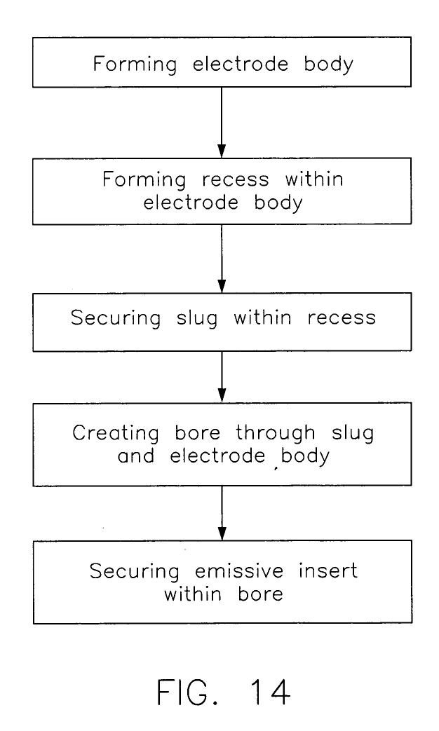

Although the emissive insert 38 is preferably secured within the bore 54 after the faces 50 and 52 are formed, it should be understood that the faces 50 and 52 can be formed after the emissive insert 38 is secured within the bore 54 while still remaining within the scope of the present invention. The specific steps of alternate manufacturing processes in accordance with the teachings of the present invention are illustrated in detail in FIGS. 12-14, wherein alternate low cost methods for creating the hybrid electrode 20 are laid out in a step-by-step fashion. It should be understood that the order of the steps shown is merely illustrative of the preferred forms of the present invention and that changing the order of the steps would result in manufacturing methods that remain within the scope of the present invention.

Referring more specifically to FIG. 9, the electrode body 22 further comprises a side wall 70 that defines an edge 72 as shown. Accordingly, the face 50 preferably extends between the side wall 70 and the edge 72. As further shown, the slug 42 is joined to the electrode body 22 along an interface 74, wherein the interface 74 is preferably positioned distally from the edge 72 to improve the reliability of the connection between the slug 42 and the electrode body 22. It should be understood that the interface 74 may alternately be positioned proximally from the edge 72 while remaining within the scope of the present invention, thereby positioning the braze at the interface 74 away from the edge 72 to reduce the possibility of arcing at the interface 74. As further shown, the slug 42 (or secondary body 32 in the final geometry of the hybrid electrode 20) also defines a radiused surface 76 that extends between the secondary face 52 and the distal face 34 in one form of the present invention.

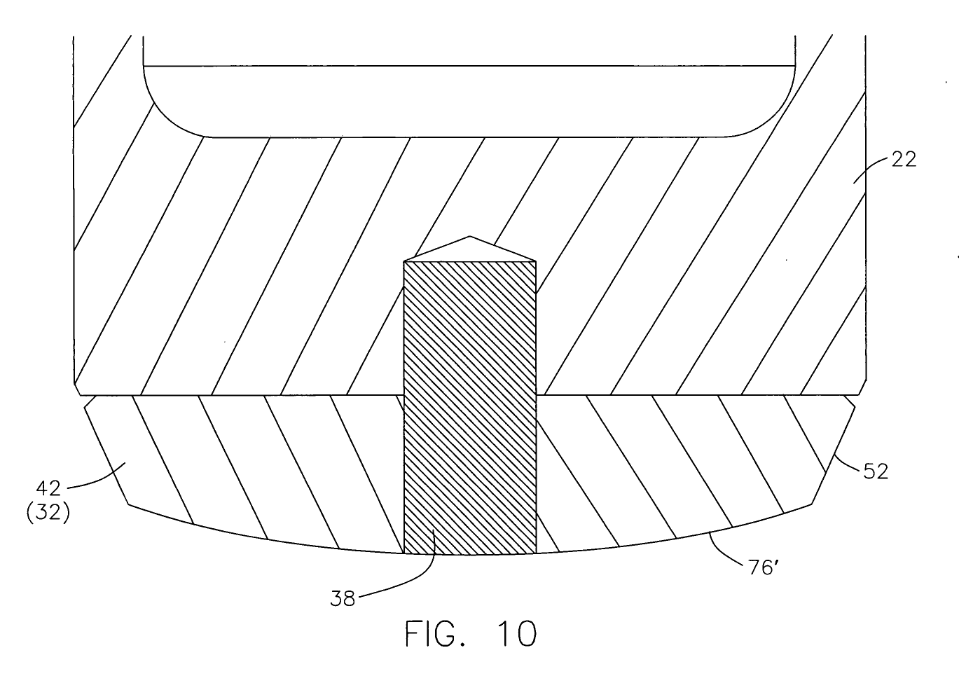

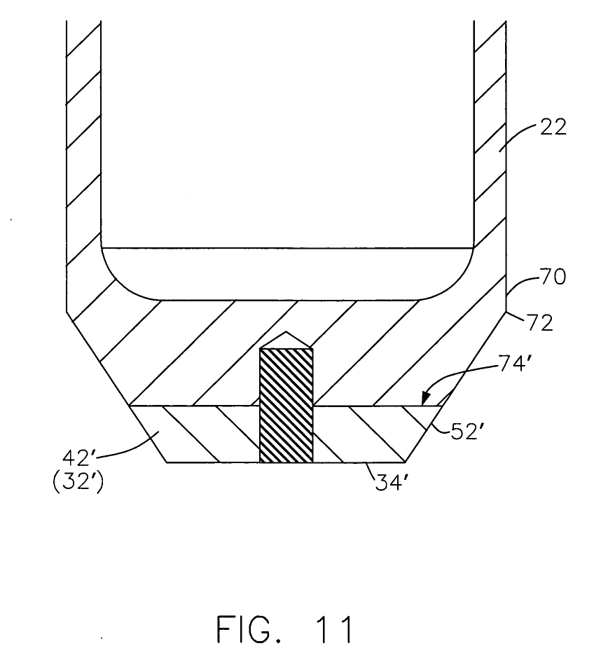

In another form of the present invention, as illustrated in FIG. 10, the radiused surface 76′ extends all the way from the secondary face 52 to the emissive insert 38, resulting in yet another alternate geometry for the secondary body 32. It should be understood that additional variations on the geometry of the secondary body 32 and the electrode body 22 may also be employed while remaining within the scope of the present invention. For example, in another form of the present invention as shown in FIG. 11, the secondary face 52′ of the secondary body 32′ extends from the side wall 70 of the electrode body 22 all the way to the distal face 34′ of the secondary body 32′. Preferably the interface 74′ is again positioned distally from the edge 72 of the electrode body 22 to provide a more robust connection between the secondary body 32 and the electrode body 22. It should be understood that the interface 74′ may alternately be positioned proximally from the edge 72 while remaining within the scope of the present invention, thereby positioning the braze at the interface 74′ away from the edge 72 to reduce the possibility of arcing at the interface 74′.

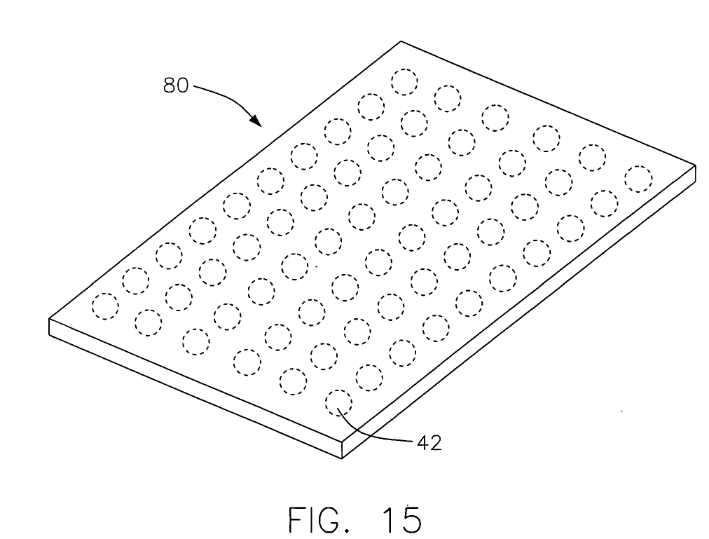

In yet another form of the present invention, the slug 42 is formed by a coining process to further reduce costs of the manufacturing process. (Coining processes are known in the art and are not described in detail herein for purposes of clarity.) As shown in FIG. 15, sheet stock 80 is employed with a coining machine (not shown), wherein the individual slugs 42 are coined from the sheet stock 80. Accordingly, the costs of the individual slugs 42 are relatively low when formed by a coining process when the hybrid electrodes 20 are to be mass produced.

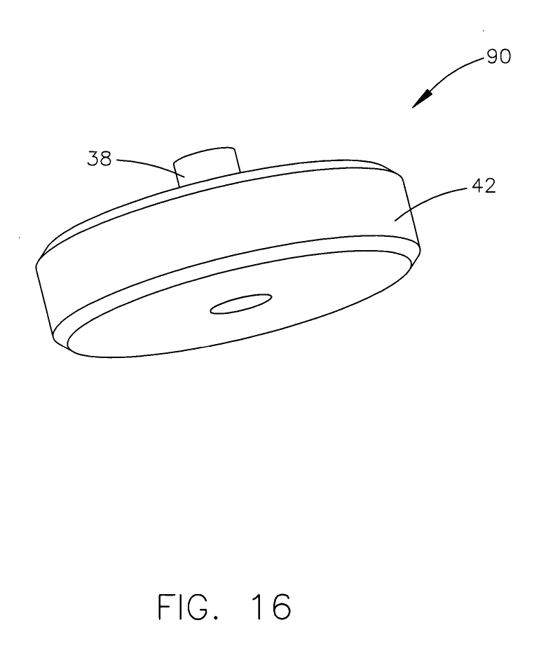

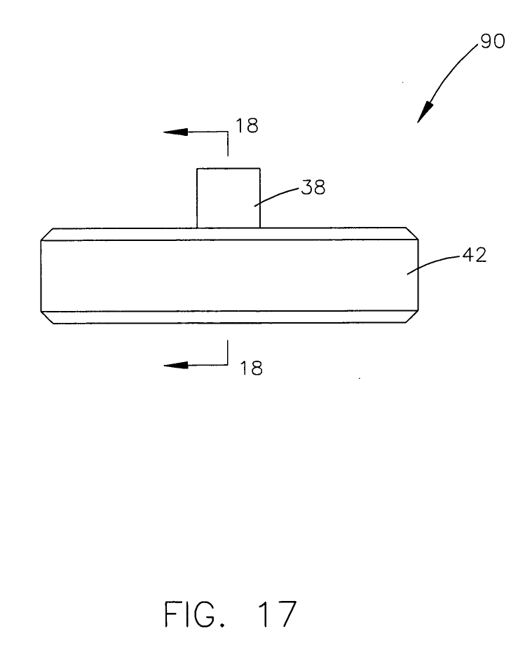

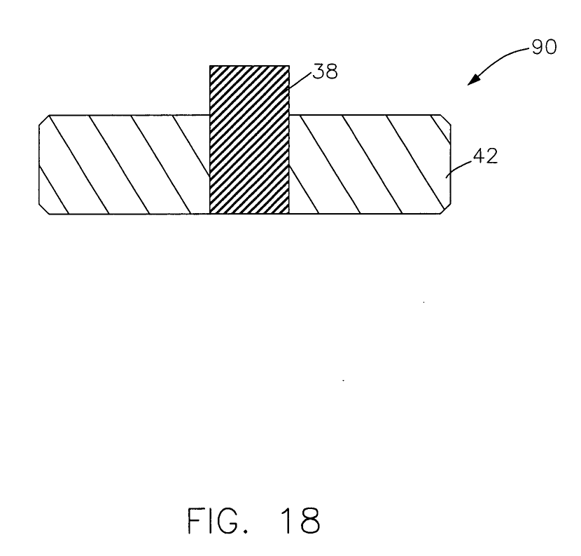

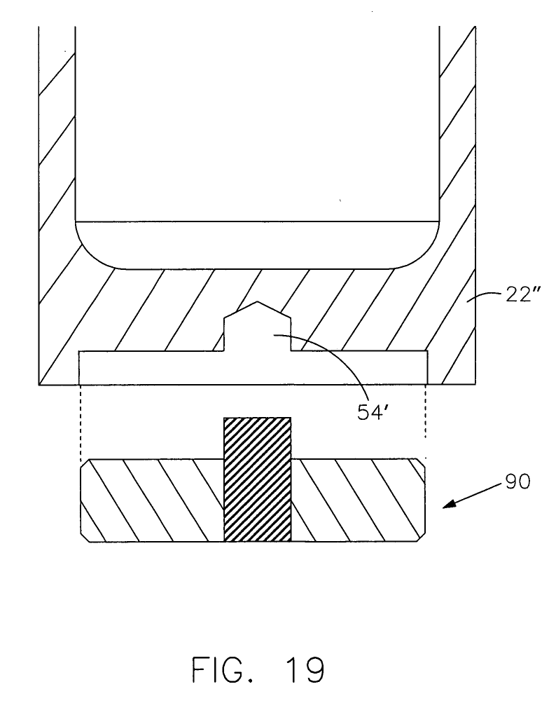

In another form, the coining process as illustrated and described herein includes pressing the emissive element 38 into the slug 42 during the coining process, thereby resulting in an electrode subassembly 90 as illustrated in FIGS. 16 through 19. The electrode subassembly 90 comprises the slug 42 and the emissive insert 38, preformed and ready to be secured to the electrode body 22 as shown in FIG. 19. Accordingly, the electrode body 22 is prepared with a bore 54′ prior to insertion of the electrode subassembly 90. The electrode subassembly 90 is then secured within the recess 40 according to the methods described herein. Additionally, the slug 42 is shaped to the geometries as described herein to form the secondary body 32 of the hybrid electrode 20.

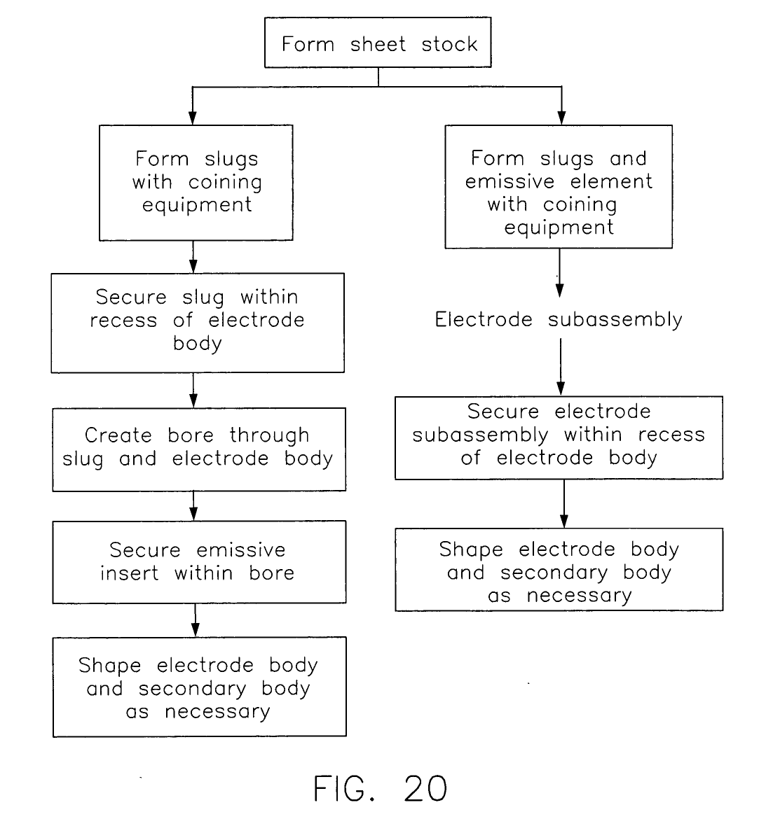

Referring to FIG. 20, the alternate method of forming a hybrid electrode 20 using a coining process is illustrated in greater detail. As shown, the sheet stock 80 is first formed, and then the slugs 42 are formed by the coining equipment in a predetermined geometry. In the alternate form, the emissive insert 38 is simultaneously coined with the slug 42 to form the electrode subassembly 90. The slug 42 or the electrode subassembly 90, depending on the method, are then secured within the recess 40 of the electrode body 22 and the hybrid electrode 20 is further processed in accordance with the manufacturing steps as previously described herein.

Accordingly, various low cost methods of forming hybrid electrodes that provide increased life are provided by the teachings of the present invention. It should be understood that the description of the invention is merely exemplary in nature and, thus, variations that do not depart from the substance of the invention are intended to be within the scope of the invention. Such variations are not to be regarded as a departure from the spirit and scope of the invention.

What is claimed is:

1. A method of fabricating an electrode for use in a plasma arc torch comprising:
forming an electrode body having a proximal end portion and a distal end portion;
forming a recess within the distal end portion of the electrode body;
brazing a slug within the recess of the electrode body;
forming at least one face around the distal end portion of the electrode and a distal end face of the slug;
creating a bore through the distal end face of the slug such that the bore extends through the slug and the electrode body; and
securing an emissive element within the bore, the emissive element being in contact with the slug and the electrode body.

2. The method according to claim 1, wherein the electrode body comprises a metal selected from the group consisting of copper and copper alloys.

3. The method according to claim 1, wherein the slug comprises a metal selected from the group consisting of silver, silver alloys, gold, and gold alloys.

4. The method according to claim 1, wherein the slug comprises a silver alloy with an additional material selected from the group consisting of copper, aluminum, iron, lead, zinc, and alloys thereof.

5. The method according to claim 1, wherein the emissive element comprises a metal selected from the group consisting of hafnium, zirconium, tungsten and alloys thereof.

6. The method according to claim 1, wherein the emissive element is secured within the bore by press fitting.

7. The method according to claim 1, wherein the brazing comprises applying a compound selected from the group consisting of silver solder paste, silver alloy solder paste, a flux and silver solder paste, and a flux.

8. The method according to claim 1, wherein the face is formed by applying a process selected from the group consisting of machining, metal injection molding, casting, stamping, and molding.

9. The method according to claim 1, wherein the recess is treated by a method selected from the group consisting of alcohol washing and drying, physical vapor deposition, chemical vapor deposition, anodizing, and plating.

10. The method according to claim 1, further comprising forming a secondary face around the distal end face of the slug.

11. The method according to claim 1, wherein the face and a side wall of the electrode define an edge, and the slug is brazed distally from the edge.

12. A method of fabricating an electrode for use in a plasma arc torch, comprising:
forming an electrode body having a proximal end portion and a distal end portion;
forming a cavity within the proximal end portion of the electrode body;
forming a recess within the distal end portion of the electrode body;
brazing a slug within the recess of the electrode body;
machining the slug and the electrode body to form at least one face around the distal end portion of the electrode body and the slug;
creating a bore through a distal face of the slug such that the bore is at least partially exposed to the slug and the electrode body; and
fixedly mounting an emissive element within the bore, the emissive element being in contact with the slug and the electrode body and not with the cavity.

13. The method according to claim 11, further comprising forming a secondary face around the distal face of the slug.

14. A method of forming an electrode for use in a plasma arc torch comprising:
forming an electrode body defining a proximal end portion and a distal end portion;
forming a recess within the distal end portion;
securing a slug within the recess of the distal end portion;
creating a bore through the slug and the distal end portion of the electrode body; and
securing an emissive insert within the bore.

15. The method according to claim 14, wherein the slug is secured within the recess by a process selected from the group consisting of brazing, mechanical press fitting and swaging, friction welding, mechanical threading, heat treatment, and adhesive bonding.

16. The method according to claim 14, further comprising forming at least one face around the slug.

17. The method according to claim 14, further comprising forming at least one face around the distal end portion of the electrode and the slug.

18. The method according to claim 17, further comprising forming a secondary face around the slug.

19. The method according to claim 14, further comprising forming a cavity within the proximal end portion, wherein the emissive insert is not exposed to the cavity.

20. The method according to claim 14, further comprising forming a cavity within the proximal end portion, wherein the emissive insert is exposed to the cavity.

21. A method of forming an electrode comprising the step of coining a slug, the slug being adapted for attachment to an electrode body.

22. The method according to claim 21, wherein the step of coining includes pressing an emissive element within a bore of the slug to form an electrode subassembly.

23. The method according to claim 22, wherein the electrode subassembly is secured to a distal end portion of an electrode body to form the electrode.

24. The method according to claim 23, wherein the electrode subassembly is secured to the distal end portion of the electrode body by brazing.

25. A hybrid electrode for use in a plasma arc torch comprising:
an electrode body defining a proximal end portion and a distal end portion, the electrode body comprising a cavity within the proximal end portion;
a secondary body disposed at the distal end portion of the electrode body, the secondary body defining a distal face and a bore formed through the distal face; and
an emissive element disposed within the bore and in contact with the secondary body and the electrode body, wherein the secondary body is not exposed to the cavity.

26. The hybrid electrode according to claim 25, further comprising at least one face formed around the distal end portion of the electrode body and the secondary body.

27. The hybrid electrode according to claim 26, further comprising a secondary face formed around the secondary body.

28. The hybrid electrode according to claim 26, wherein the face and a side wall of the electrode body define an edge, and an interface between the secondary body and the electrode body is positioned distally from the edge.

29. The hybrid electrode according to claim 26, wherein the face and a side wall of the electrode body define an edge, and an interface between the secondary body and the electrode body is positioned proximally from the edge.

30. The hybrid electrode according to claim 25, wherein the electrode body comprises a metal selected from the group consisting of copper and copper alloys.

31. The hybrid electrode according to claim 25, wherein the secondary body comprises a metal selected from the group consisting of silver, silver alloys, gold, and gold alloys.

32. The hybrid electrode according to claim 31, wherein the secondary body comprises a silver alloy with an additional material selected from the group consisting of copper, aluminum, iron, lead, zinc, and alloys thereof.

33. The hybrid electrode according to claim 25, wherein the emissive element is exposed to the cavity.

34. The hybrid electrode according to claim 25, wherein the emissive element is not exposed to the cavity.
